(19) United States
(12) Patent Application Publication
(10) Pub. No.: US 2025/0325832 A1
(43) Pub. Date: Oct. 23, 2025

(54) LIGHT-EMITTING HYGIENE DEVICE COMPRISING PCB DIRECTLY ASSEMBLED TO BODY

(71) Applicant: Hee Chan CHA, Seoul (KR)

(72) Inventor: Hee Chan CHA, Seoul (KR)

(21) Appl. No.: 18/864,836

(22) PCT Filed: May 9, 2023
(86) PCT No.: PCT/KR2023/006242

§ 371 Date: Nov. 11, 2024

(30) Foreign Application Priority Data
May 9, 2022 (KR) 10-2022-0056844

(51) Int. Cl. A61N 5/06; A46B 9/04; A46B 13/02; A46B 15/00; A46D 1/00; A61C 17/22; A61H 13/00; A61H 23/02; A61N 5/067

(52) U.S. Cl. CPC A61N 5/0624; A46B 9/04; A46B 13/023; A46B 15/0034; A46D 1/0207; A61C 17/222; A61C 17/225; A61H 13/00; A61H 23/0254; A61N 5/0603; A61N 5/067; A61H 2201/0153; A61H 2201/10; A61H 2201/1215; A61H 2205/02; A61N 2005/0606; A61N 2005/063; A61N 2005/0644; A61N 2005/0651; A61N 2005/0663

(57) ABSTRACT

The present invention relates to a light irradiation hygiene device with a PCB directly assembled into the main body. The device includes a main body housing the handle and head sections, a PCB configured to match the main body's shape, a power button in the handle section, a light irradiation unit with multiple light sources, a vibration motor attached via soldering, and a toothbrush head plate. The structure reduces noise and improves vibration stability, increasing convenience for users. The one-piece structure of the PCB enhances cost competitiveness, assembly efficiency, and durability, while minimizing failure points due to the removal of connecting parts. Attaching the vibration motor to the PCB, which matches the main body's shape, reduces the number of vibrating parts, decreasing noise, and simplifying the manufacturing process, leading to cost reduction and improved productivity.

### TECHNICAL FIELD

The present invention relates to a light irradiation hygiene device equipped with a Printed Circuit Board (PCB) directly assembled into the main body, and more specifically, to a light irradiation hygiene device that comprises an electric toothbrush, where the PCB with a vibration motor attached can be directly assembled into the ribs inside the main body.

### BACKGROUND ART

Generally, to hygienically manage teeth, methods such as brushing with a toothbrush to remove oral bacteria that cause plaque and tartar formation, or using mouthwash with chemical antibacterial agents after brushing, are commonly employed. As a method to effectively brush teeth, electric toothbrushes have been widely introduced. Additionally, since regular brushing alone is not sufficient to effectively remove bacteria, electric toothbrushes that irradiate light of a specific wavelength are also disclosed.

However, chemical removal methods using oral cleansers are generally the only approach used, and while electric toothbrushes that emit light—such as those that irradiate white light, as well as blue and red LED electric toothbrushes—have been recently released in large numbers, there has been a limitation in that no specific method for effectively inhibiting oral bacteria has been proposed. In particular, conventional electric toothbrushes are configured with a vibration motor inside and an antibacterial function that irradiates light. However, the vibration motor is placed in a plastic housing and is physically separated from the circuit board during assembly. This results in the typical configuration of three separate PCBs: a circuit board where the light source is formed, a circuit board where the vibration motor is connected, and a circuit board where the battery is connected. These conventional electric toothbrushes, with their three-piece PCB structure, lead to increased costs and manufacturing processes, as well as problems with increased noise and decreased vibration stability during use. Korean Patent Publication No. 10-0773210 is disclosed as prior art.

### Invention Content, Problems to be Solved

The present invention has been proposed to solve the problems of the methods previously suggested. Specifically, it aims to provide a light irradiation hygiene device that includes a main body forming the outer housing appearance of the handle portion and the head portion. The PCB is configured to correspond to the shape of the main body and is arranged inside the main body, with a power button formed in the handle portion and a light source unit that includes multiple light sources irradiating light through a waveguide in the head portion. A vibration motor is attached to the PCB through soldering, placed at the top of the handle portion's upper region. The brush head is fastened to the upper part of the light source unit in the head portion of the PCB and includes at least one toothbrush bristle formed through a waveguide. By configuring the PCB with an attached vibration motor to be directly assembled into the ribs of the main body, the invention reduces noise caused by the PCB-attached vibration motor, decreases noise through vibration stability, and improves user convenience by increasing vibration stability. The goal is to provide a light irradiation hygiene device with a PCB directly assembled into the main body.

The present invention has been proposed to solve the problems of the methods previously suggested. Specifically, it aims to provide a light irradiation hygiene device that includes a main body forming the outer housing appearance of the handle portion and the head portion. The PCB is configured to correspond to the shape of the main body and is arranged inside the main body, with a power button formed in the handle portion and a light source unit that includes multiple light sources irradiating light through a waveguide in the head portion. A vibration motor is attached to the PCB through soldering, placed at the top of the handle portion's upper region. The brush head is fastened to the upper part of the light source unit in the head portion of the PCB and includes at least one toothbrush bristle formed through a waveguide. By configuring the PCB with an attached vibration motor to be directly assembled into the ribs of the main body, the invention reduces noise caused by the PCB-attached vibration motor, decreases noise through vibration stability, and improves user convenience by increasing vibration stability. The goal is to provide a light irradiation hygiene device with a PCB directly assembled into the main body.

In addition, the invention proposes to configure the conventional PCB structure—comprised of multiple or split components—into a one-piece structure. By directly assembling the PCB into the ribs inside the main body, the invention improves cost competitiveness through part reduction, enhances assembly efficiency and speed, and increases durability by minimizing failure points from disconnected components.

Furthermore, the invention proposes to attach the vibration motor to the PCB and ensure that the PCB is assembled in a way that it directly contacts the external shape of the main body. This configuration reduces the number of vibrating parts, cutting down noise. Additionally, it simplifies the manufacturing process, reduces costs, and improves productivity. The goal is to provide a light irradiation hygiene device with a PCB directly assembled into the main body.

The light irradiation hygiene device with a PCB directly assembled into the main body, according to the features of the present invention, includes a main body that forms the outer housing appearance of the handle portion and the head portion. The PCB is configured to correspond to the shape of the main body and is arranged inside the main body, with a power button formed in the handle portion and a light source unit in the head portion that includes multiple light sources irradiating light through a waveguide. A vibration motor is placed in the upper region of the handle portion of the main body and is attached to the PCB via soldering. The brush head, fastened to the upper part of the light source unit in the head portion of the PCB, includes at least one toothbrush bristle formed through a waveguide. The PCB with the attached vibration motor is configured to be directly assembled into the ribs inside the main body.

Preferably, the PCB is configured as a one-piece structure that can be arranged inside the main body to correspond to the shape of the main body. In this configuration, the PCB is directly assembled into the ribs inside the main body, improving assembly efficiency and speed. By eliminating connecting components due to the direct assembly into the main body, the durability can be enhanced.

Preferably, the PCB can be further configured to include a battery connector formed inside the handle portion of the main body.

Preferably, the light source unit of the PCB includes a first light source that irradiates short-wavelength light and a second light source that irradiates long-wavelength light. The multiple light sources can be configured as a set that includes at least one first light source irradiating short-wavelength light and a set that includes at least one second light source irradiating long-wavelength light.

More preferably, the light source unit can be configured with a structure where multiple light sources are arranged in a repeating pattern.

More preferably, the light source unit includes a first light source that irradiates short-wavelength light and a second light source that irradiates long-wavelength light, where the short-wavelength light irradiated from the first light source is blue light, and the long-wavelength light irradiated from the second light source is red light.

More preferably, the light source unit can be composed of the first and second light sources as LED (Light-Emitting Diode) or laser diodes.

According to the light irradiation hygiene device with a PCB directly assembled into the main body, as proposed in the present invention, the device comprises a main body that forms the outer housing appearance of the handle portion and the head portion. The PCB, configured to correspond to the shape of the main body, is arranged inside the main body, with a power button formed in the handle portion and a light source unit in the head portion that includes multiple light sources irradiating light through a waveguide. A vibration motor is placed in the upper region of the handle portion of the main body and is attached to the PCB via soldering. The brush head, fastened to the upper part of the light source unit in the head portion of the PCB, includes at least one toothbrush bristle formed through a waveguide. The PCB, with the attached vibration motor, is configured to be directly assembled into the ribs inside the main body. This configuration reduces noise caused by the PCB-attached vibration motor, decreases noise through vibration stability, and enhances user convenience by increasing vibration stability.

Furthermore, according to the light irradiation hygiene device with a PCB directly assembled into the main body, as proposed in the present invention, the PCB is configured as a one-piece structure instead of multiple or divided structures. The PCB is directly assembled into the ribs inside the main body, which not only improves cost competitiveness due to reduced components but also enhances assembly efficiency and speed. Additionally, by eliminating connecting components through direct assembly into the outer body, the durability is further increased by minimizing potential failure points.

Moreover, according to the light irradiation hygiene device with a PCB directly assembled into the main body, as proposed in the present invention, the vibration motor is attached to the PCB, and the PCB is configured to be assembled in a structure where it contacts the outer shape of the main body. This reduces the number of vibrating parts, thereby decreasing noise, and simplifies the manufacturing process, which in turn can reduce costs and improve productivity.

### BRIEF DESCRIPTION OF THE DRAWINGS

FIG. 1 is a diagram illustrating the configuration of a light irradiation hygiene device with a PCB directly assembled into the main body, according to an embodiment of the present invention, shown as a functional block.

FIG. 2 is a diagram illustrating the configuration of the main body of the light irradiation hygiene device with a PCB directly assembled into the main body, according to an embodiment of the present invention, shown as a functional block.

FIG. 3 is a diagram illustrating the configuration of the PCB of the light irradiation hygiene device with a PCB directly assembled into the main body, according to an embodiment of the present invention, shown as a functional block.

FIG. 4 is a perspective view illustrating the overall configuration of the light irradiation hygiene device with a PCB directly assembled into the main body, according to an embodiment of the present invention.

FIG. 5 is a perspective view illustrating the configuration of the main body of the light irradiation hygiene device with a PCB directly assembled into the main body, according to an embodiment of the present invention.

FIG. 6 is a front view of the main body of the light irradiation hygiene device with a PCB directly assembled into the main body, according to an embodiment of the present invention.

FIG. 7 is a diagram illustrating the front and rear configurations of the PCB of the light irradiation hygiene device with a PCB directly assembled into the main body, according to an embodiment of the present invention.

FIG. 8 is a diagram illustrating the relationship between the light source unit and the brush head in the light irradiation hygiene device with a PCB directly assembled into the main body, according to an embodiment of the present invention.

FIG. 9 is a diagram illustrating the configuration of the light irradiation hygiene device with a PCB directly assembled into the main body, showing the light source unit and the brush head in a coupled state, according to an embodiment of the present invention.

FIG. 10 is a top view illustrating the configuration of the brush head of the light irradiation hygiene device with a PCB directly assembled into the main body, according to an embodiment of the present invention.

### SPECIFIC DETAILS FOR IMPLEMENTING THE INVENTION

Below, with reference to the attached drawings, a preferred embodiment of the present invention is described in detail so that a person skilled in the art to which the invention pertains can easily carry out the invention. However, in describing the preferred embodiment of the present invention in detail, if it is determined that a detailed explanation of related known functions or configurations may unnecessarily obscure the subject matter of the invention, such detailed explanations are omitted. Additionally, the same reference numerals are used throughout the drawings for parts that have similar functions and actions.

Additionally, throughout the specification, when it is stated that one part is “connected” to another, this includes not only cases where the parts are “directly connected,” but also cases where they are “indirectly connected” through other components in between. Furthermore, when a component is said to “include” another, unless specifically stated otherwise, it means that other components may also be included, rather than excluding other components.

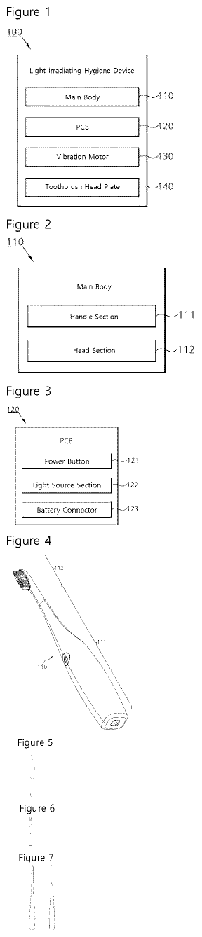

FIG. 1 is a functional block diagram showing the configuration of the light irradiation hygiene device with a PCB directly assembled into the body according to an embodiment of the present invention. FIG. 2 is a functional block diagram showing the configuration of the body part of the light irradiation hygiene device with a PCB directly assembled into the body according to an embodiment of the present invention. FIG. 3 is a functional block diagram showing the configuration of the PCB of the light irradiation hygiene device with a PCB directly assembled into the body according to an embodiment of the present invention. FIG. 4 is a perspective view showing the general configuration of the light irradiation hygiene device with a PCB directly assembled into the body according to an embodiment of the present invention. FIG. 5 is a perspective view showing the general configuration of the body part of the light irradiation hygiene device with a PCB directly assembled into the body according to an embodiment of the present invention. FIG. 6 is a front view showing the general configuration of the body part of the light irradiation hygiene device with a PCB directly assembled into the body according to an embodiment of the present invention. FIG. 7 is a diagram showing the front and rear configurations of the PCB of the light irradiation hygiene device with a PCB directly assembled into the body according to an embodiment of the present invention. As shown in FIGS. 1 to 7, the light irradiation hygiene device with a PCB directly assembled into the body (100) according to an embodiment of the present invention may include a body part (110), PCB (120), a vibration motor (130), and a toothbrush head plate (140).

The body part (110) is the structure that forms the housing exterior of the handle part (111) and the head part (112). The handle part (111) of the body part (110) is the part where the user holds the toothbrush, and the power button (121) may be exposed here. The head part (112) of the body part (110) is where the waveguide (to be described later), consisting of the toothbrush bristles (141) and massage bristles (142), and the light source part (122) are arranged. The light emitted from the light source can be transmitted to the user's teeth or gums through the toothbrush bristles (141).

The PCB (120) is designed in a shape corresponding to the form of the body part (110) and is arranged inside the body part (110). It includes a power button (121) in the handle area and a light source unit (122) in the head area, which contains multiple light sources that emit light through the waveguide. As shown in FIGS. 6 and 7, the PCB (120) is configured as a one-piece structure that can be arranged inside the body part (110) to correspond with its shape. This structure enhances the ease of assembly and assembly speed, as well as improving durability by eliminating connecting components when assembled directly into the body part (110). Additionally, the PCB (120) is designed as a one-piece structure, unlike the traditional multi-piece or segmented PCB, which simplifies the manufacturing process and reduces costs.

Additionally, the PCB (120) with the attached vibration motor (130) can be directly assembled into the internal ribs of the body part (110). This assembly structure can enhance cost competitiveness by reducing the number of components. Moreover, the direct assembly of the PCB (120) with the vibration motor (130) into the body part (110) eliminates connecting components, which improves durability by reducing potential failure points. It also reduces the number of parts that vibrate due to the motor, leading to a reduction in noise. The direct assembly into the body part (110) enhances both assembly efficiency and speed, as well as improving vibration transmission efficiency.

Additionally, the PCB (120) can be configured to include a battery connector (123) formed inside the handle portion (111) of the body part (110). Furthermore, the light source unit (122) of the PCB (120) includes a first light source (122a) that emits short-wavelength light and a second light source (122b) that emits long-wavelength light. The multiple light sources can be structured into sets, with one set containing at least one first light source (122a) that emits short-wavelength light, and another set containing at least one second light source (122b) that emits long-wavelength light.

Additionally, the light source unit (122) can be structured such that multiple light sources are arranged in a repeating pattern. This light source unit (122) includes a first light source (122a) that emits short-wavelength light and a second light source (122b) that emits long-wavelength light. The short-wavelength light emitted by the first light source (122a) may be blue light, while the long-wavelength light emitted by the second light source (122b) may be red light. Furthermore, the light source unit (122) can also include ultraviolet, infrared, or other non-visible light from the non-visible spectrum, or it could emit yellow or green light in addition to red or blue.

Furthermore, the light source unit (122) may be configured such that the first light source (122a) and the second light source (122b) are composed of LEDs (Light-Emitting Diodes) or laser diodes.

The vibration motor (130) is placed in the upper region of the handle portion (111) of the main body (110) and is attached to the PCB (120) through soldering. By configuring the vibration motor (130) as an attachment structure to the PCB (120), noise resulting from vibrations during the use of the electric toothbrush is reduced, and the stability of the vibrations is enhanced. Specifically, the attachment of the motor to the PCB (120), as opposed to a separate motor configuration, contributes to reduced noise and improved vibration stability, thereby enhancing user convenience. The vibration motor (130) generates vibrations in the light irradiation hygiene device (100), enabling the electric toothbrush to perform its intended function.

The toothbrush head plate (140) is positioned above the light source section (122) formed in the head portion of the PCB (120) and is secured in place. It includes at least one toothbrush bristle (141) formed as a waveguide. The bristle (141) formed as a waveguide can be made of optical fibers. This configuration allows the light from the light source to pass through the waveguide and reach the bristles, which can then deliver the light to the user's teeth and gums during use.

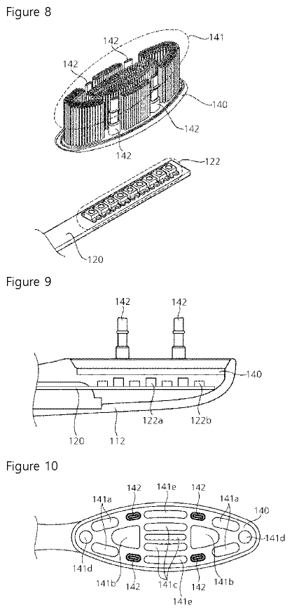

As shown in FIGS. 8 to 10, the toothbrush bristles (141) include at least one waveguide and emit light that is irradiated from the light source. A waveguide refers to a tube with the property of transmitting light generated by the light source. The bristles (141) convey the light from the light source to the user's teeth, and the bristles that use a waveguide may be constructed from optical fibers. In this configuration, the light from the light source is efficiently transmitted through the waveguide, allowing it to be directed to the desired areas during use.

Additionally, the massage bristles (142) may also be constructed using a waveguide. The massage bristles (142) are used for massaging the user's gums and can transmit light from the light source to massage areas such as the gums, the inside of the cheeks, or the tongue. To effectively transmit light from the light source, the massage bristles (142) may be made from a bundle of optical fibers. Since optical fibers must have a diameter that is not too large to effectively transmit light, the massage bristles (142) may form a set (or bundle) of optical fibers, and these bundles can be inserted into the capsule of the massage bristles (142) as a way of integrating them. This construction ensures that light is efficiently transmitted to the target areas during use.

Additionally, the toothbrush bristles (141) and massage bristles (142) formed on the toothbrush head plate (140) can be detachable from the head part (112) of the main body (110), allowing for easy replacement depending on usage. This feature enables the user to replace the brush head with different types of bristles or heads, enhancing the versatility and longevity of the device.

Additionally, as shown in FIG. 10, the toothbrush bristles (141) can be configured such that the bristles located at both ends of the toothbrush head plate (140), denoted as (141d), are longer than the other bristles (141a, 141b, 141c, 141e). This arrangement allows the bristles to better focus light on the molars, ensuring more effective light irradiation and improved cleaning performance in areas that are harder to reach.

Furthermore, the toothbrush bristles (141a, 141b, 141c, 141e) can be configured in a rectangular shape according to the arrangement of the light sources. The massage bristle (142) can be positioned around the center of the elliptical-shaped toothbrush head plate (140), placed at the virtual rectangular vertices surrounding the outer perimeter. This positioning allows the massage bristles (142) to effectively massage the gums by focusing on specific areas, such as the inside of the cheeks or the tongue, while also transmitting light from the light sources.

In addition, the light source unit (122) can emit blue light to provide oral sterilization or teeth whitening effects within the user's mouth, and red light can be emitted to produce effects such as anti-inflammatory properties and collagen formation. Furthermore, the multiple light sources of the first light source (122a) and second light source (122b) can be arranged alternately, which allows for enhanced light intensity through constructive interference of the light, thus improving the effectiveness of the light therapy.

As described above, the light irradiation hygiene device according to an embodiment of the present invention, which includes a PCB assembled directly to the main body, comprises: a main body that forms the housing appearance of the handle part and head part; a PCB shaped to correspond to the shape of the main body, which is disposed inside the main body, with a power button formed in the handle part and a light source unit including a plurality of light sources that irradiate light through a waveguide in the head part; a vibration motor attached to the PCB and disposed at the top of the handle part of the main body, with the PCB mounted directly to the internal ribs of the main body. By employing a vibration motor attached to the PCB, noise is reduced, and the stability of the vibration is enhanced, improving convenience during use. Additionally, by configuring the PCB in a one-piece structure rather than multiple or segmented pieces, the structure of the PCB being directly assembled to the internal ribs of the main body improves cost competitiveness through component reduction, increases assembly efficiency and speed, and enhances durability by eliminating connecting components and minimizing potential failure points. Specifically, by attaching the vibration motor to the PCB and assembling it in such a way that the PCB touches the exterior of the main body, the number of vibrating components is reduced, noise is minimized, and the manufacturing process is simplified, resulting in cost reduction and improved productivity.

As described above, the present invention can be subject to various modifications and applications by those skilled in the art to which the invention pertains. The scope of the technical idea according to the present invention should be defined by the claims below.

### DESCRIPTION OF REFERENCE NUMBERS

100: Light irradiation hygiene device according to an embodiment of the present invention
110: Main body
111: Handle section
112: Head section
120: PCB (Printed Circuit Board)
121: Power button
122: Light source section
122a: First light source
122b: Second light source
123: Battery connector
130: Vibration motor
140: Toothbrush head plate
141: Toothbrush bristles
141a, 141b, 141c, 141d, 141e: Toothbrush bristles
142: Massage bristle

1: A light irradiation hygiene device comprising a PCB (Printed Circuit Board) directly assembled into the main body, the device comprising: a main body section forming the housing exterior of the handle section and the head section; a PCB configured in a shape corresponding to the form of the main body section, disposed inside the main body section, with a power button formed in the handle section area and a light source section including a plurality of light sources configured to irradiate light via a waveguide in the head section area; a vibration motor disposed in the upper area of the handle section of the main body section and attached to the PCB via soldering; and a toothbrush head plate located above the light source section formed in the head section area of the PCB and secured thereto, the toothbrush head plate including at least one toothbrush bristle formed by a waveguide, wherein the PCB with the attached vibration motor is directly assembled into the internal rib (Rib) of the main body section.

2: The light irradiation hygiene device according to claim 1, wherein the PCB is configured as a one-piece structure that can be disposed inside the main body section in a shape corresponding to the form of the main body section, and the PCB is directly assembled into the internal rib (Rib) of the main body section, thereby enhancing the assemblability and assembly speed, and improving durability by eliminating connecting parts due to direct assembly into the main body.

3: The light irradiation hygiene device according to claim 1, wherein the PCB further includes a battery connector (123) formed and disposed inside the handle portion of the main body section.

4: The light irradiation hygiene device according to claim 1 wherein the light source section of the PCB includes a first light source that emits short-wavelength light and a second light source that emits long-wavelength light, and the plurality of light sources is structured in sets, with at least one first light source that emits short-wavelength light and at least one second light source that emits long-wavelength light.

5: The light irradiation hygiene device according to claim 4, wherein the light source section is structured such that a plurality of light sources are arranged in a repetitive pattern.

6: The light irradiation hygiene device according to claim 4, wherein the light source section includes a first light source that irradiates short-wavelength light, and a second light source that irradiates long-wavelength light, wherein the short-wavelength light irradiated by the first light source is blue light, and the long-wavelength light irradiated by the second light source is red light.

7: The light irradiation hygiene device according to claim 4, wherein the light source section is characterized in that the first light source and the second light source are composed of LED (Light-Emitting Diode) or laser diodes.

8: The light irradiation hygiene device according to claim 4, wherein the toothbrush head plate includes at least one toothbrush formed by a waveguide, and the toothbrush formed by the waveguide is made of optical fiber.
